(19) United States
(12) Patent Application Publication
(10) Pub. No.: US 2024/0180437 A1
(43) Pub. Date: Jun. 6, 2024

(54) BLOOD PRESSURE MEASUREMENT DEVICE

(71) Applicant: OMRON HEALTHCARE Co., Ltd., Kyoto (JP)

(72) Inventors: Masaki HARADA, Kyoto (JP); Yoshihide TOKKO, Kyoto (JP)

(21) Appl. No.: 18/442,579

(22) Filed: Feb. 15, 2024

Related U.S. Application Data
(63) Continuation of application No. PCT/JP2023/009120, filed on Mar. 9, 2023.

(30) Foreign Application Priority Data
Mar. 29, 2022 (JP) 2022-053720

(51) Int. Cl. A61B 5/022; A61B 5/00; A61B 5/021

(52) U.S. Cl. CPC A61B 5/02233; A61B 5/02141; A61B 5/681

(57) ABSTRACT

A blood pressure measurement device includes a cuff structure including a connecting portion provided at a portion facing a wrist side of a case, the connecting portion being connected to a connected portion of the case, a belt body including a bag body formed with a hole disposed with the connecting portion on one end side in a longitudinal direction at a portion facing the connected portion in a state where the cuff structure is disposed inside the bag body, and an attachment portion including one end portion fixed on the one end side of the bag body and the other end side from the hole of the bag body, and the other end portion attached to an attached portion, the attachment portion having a length from the one end portion to the attached portion shorter than a length from the one end portion to the connecting portion.

### CROSS-REFERENCE TO RELATED APPLICATIONS

This application is the U.S. national stage application filed pursuant to 35 U.S.C. 365(c) and 120 as a continuation of International Patent Application No. PCT/JP2023/009120, filed Mar. 9, 2023, which application claims priority to Japanese Patent Application No. 2022-053720, filed Mar. 29, 2022, which applications are incorporated herein by reference in their entireties.

### TECHNICAL FIELD

The present invention relates to a blood pressure measurement device to be worn on a wrist.

### BACKGROUND ART

In recent years, blood pressure measurement devices used for measuring a blood pressure are being used as means to check health status at home, as well as in medical facilities. A blood pressure measurement device detects vibration of the artery wall to measure blood pressure by, for example, inflating and contracting a cuff wound around the upper arm or the wrist of a living body and detecting the pressure in the cuff by using a pressure sensor.

As such a blood pressure measurement device, a so-called integral type is known in which a cuff is integrated with a device body that feeds a fluid to the cuff. Further, as the integral type blood pressure measurement device, a wearable device to be worn on a wrist is also considered. As a technique for wearing a blood pressure measurement device on a wrist, there is known a technique in which a belt provided in a device body is tightened and fixed to the wrist in a state where a cuff is disposed inside the belt.

Further, for this type of blood pressure measurement device, there is known a technique that uses a curler between a belt and a cuff, which brings the inflated cuff to close contact with a wrist. In such a blood pressure measurement device, the cuff is fixed to an inner circumferential surface of the curler. JP 2019-118418 A discloses a technique in which a curler and a cuff are fixed to a device body through a back cover, and the cuff is connected to a flow path portion that is fluidly continuous with a pump.

### CITATION LIST

### Patent Literature

Patent Document 1: JP 2019-118418 A

### SUMMARY OF INVENTION

### Technical Problem

In the above-described blood pressure measurement device to be worn on a wrist by using the belt, since the cuff and the belt are separate bodies, when a user wears the blood pressure measurement device on a wrist, the belt and the cuff may be relatively displaced in an axial direction of the wrist along a longitudinal direction of the forearm and worn on the wrist. When the belt and the cuff are relatively displaced in the axial direction of the wrist, the cuff is not suitably pressed against the wrist at the time of blood pressure measurement, which may reduce the accuracy of the blood pressure measurement.

Accordingly, an object of the present invention is to provide a blood pressure measurement device that can prevent measurement accuracy of a blood pressure from being reduced.

### Solution to Problem

According to one aspect, there is provided a blood pressure measurement device to be worn on a wrist of a user, the blood pressure measurement device including a case including a case body, an attached portion provided at one of symmetrical positions in a circumferential direction of an outer peripheral surface of the case body, a shaft portion provided at the other symmetrical position, and a connected portion provided on a wrist side of the case body, a pump accommodated in the case body, the pump being fluidly continuous with the connected portion, a cuff including a connecting portion provided at a portion facing the wrist side of the case body, the connecting portion being connected to the connected portion, the cuff being fluidly connected to the pump through the connecting portion, and a belt including a bag body inside which the cuff is accommodated, the bag body having a band shape to be wound around the wrist, the bag body being formed with a hole disposed with the connecting portion on one end side in a longitudinal direction, an attachment portion including one end portion fixed on the one end side of the bag body and the other end side from the hole, and the other end portion attached to the attached portion, the attachment portion having a length from the one end portion to the attached portion shorter than a length from the one end portion to the connecting portion disposed in the hole, and a fixing portion provided at the bag body, the fixing portion being configured to fix a portion configured to be folded back and layered at the shaft portion of the bag body.

The cuff is inflated by being supplied with a fluid, and includes a structure having a bag shape such as an air bag.

According to this aspect, the cuff is disposed in the bag body. Thus, when the blood pressure measurement device is worn on the wrist, even when the cuff moves in an axial direction of the wrist in the bag body, the cuff can be suitably pressed against the wrist because the cuff is accommodated in the bag body. As a result, measurement accuracy of a blood pressure can be improved.

There is provided a blood pressure measurement device in which one of the connected portion and the connecting portion is inserted into the other of the connected portion and the connecting portion in the blood pressure measurement device according to the one aspect described above.

According to this aspect, the connecting portion can be easily connected to the connected portion.

There is provided a blood pressure measurement device in which the attachment portion has flexibility in the blood pressure measurement device according to the one aspect described above.

According to this aspect, a movable region of the belt around the shaft portion can be enlarged. This makes it easy to attach and detach the blood pressure measurement device to and from a wrist.

There is provided a blood pressure measurement device in which the bag body and the attachment portion are made of cloth in the blood pressure measurement device according to the one aspect described above.

According to this aspect, the blood pressure measurement device can improve wearability.

### Advantageous Effects of Invention

The present invention can provide a blood pressure measurement device that can prevent measurement accuracy of a blood pressure from being reduced.

### BRIEF DESCRIPTION OF DRAWINGS

FIG. 1 is a perspective view illustrating a configuration of a blood pressure measurement device according to an embodiment of the present invention.

FIG. 2 is a side view illustrated by partially cutting the blood pressure measurement device.

FIG. 3 is a side view illustrating a configuration of a cuff structure used in the blood pressure measurement device.

FIG. 4 is a perspective view illustrating a configuration of the cuff structure and a bag used in the blood pressure measurement device.

FIG. 5 is a side view illustrated by partially cutting the configuration of the bag and the cuff structure.

FIG. 6 is a plan view illustrating the configuration of the cuff structure.

FIG. 7 is a cross-sectional view illustrating the configuration of the bag and the cuff structure.

FIG. 8 is a cross-sectional view illustrating the configuration of the bag and the cuff structure.

FIG. 9 is an explanatory diagram illustrating a state where the blood pressure measurement device is worn on a wrist.

FIG. 10 is an explanatory diagram illustrating a configuration of an attachment portion according to a modified example of the present invention.

FIG. 11 is an explanatory diagram illustrating a cuff structure according to the modified example of the present invention.

### DESCRIPTION OF EMBODIMENTS

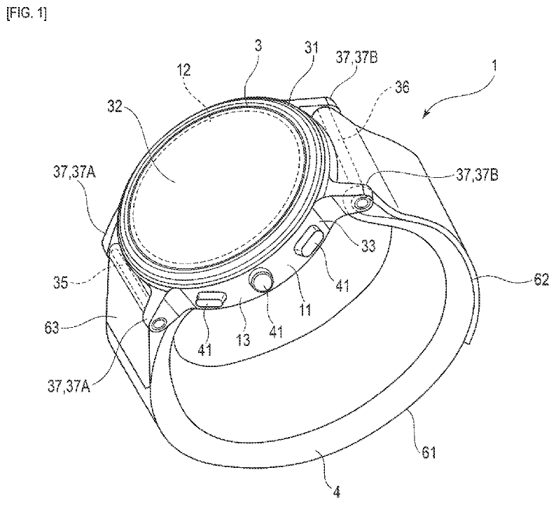

Hereinafter, an example of a blood pressure measurement device 1 according to an embodiment of the present invention will be described with reference to FIG. 1 to FIG. 9.

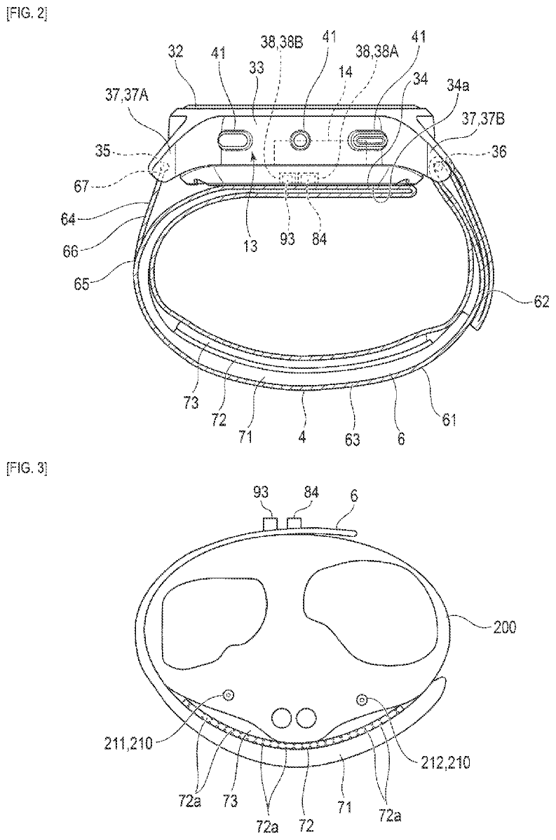

FIG. 1 is a perspective view illustrating a configuration of the blood pressure measurement device 1. FIG. 2 is a side view illustrated by partially cutting the blood pressure measurement device 1. Specifically, FIG. 2 illustrates a state in which a belt 4 used in the blood pressure measurement device 1 is cut.

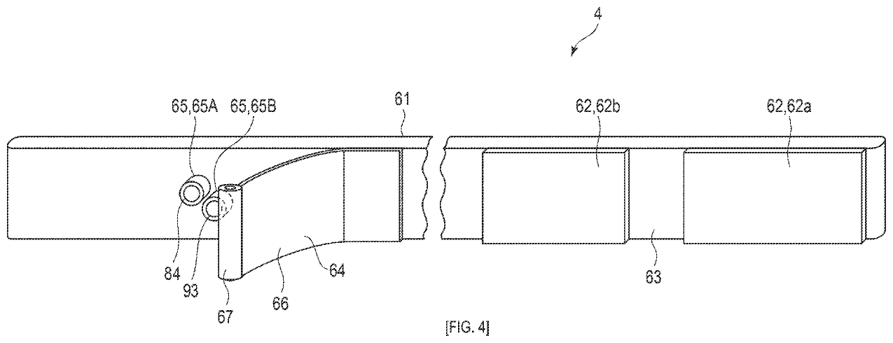

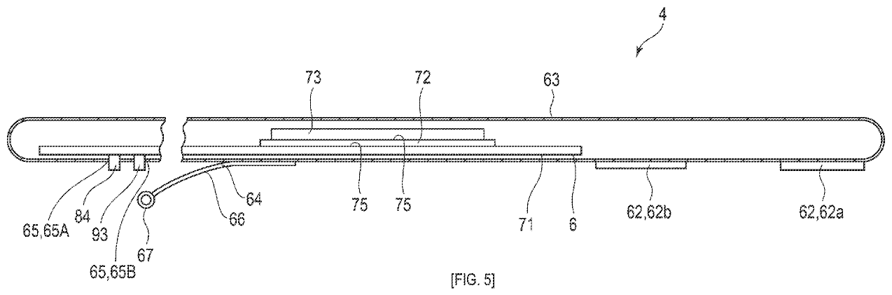

FIG. 3 is a side view illustrating a configuration of a cuff structure 6 used in the blood pressure measurement device 1. FIG. 3 illustrates a state in which the cuff structure 6 is wound around a wrist 200. FIG. 4 is a perspective view illustrating a configuration of the belt 4 accommodating the cuff structure 6. FIG. 5 is a side view illustrated by partially cutting the configuration of the belt 4 and the cuff structure 6. Specifically, FIG. 5 illustrates a state in which the belt 4 is cut.

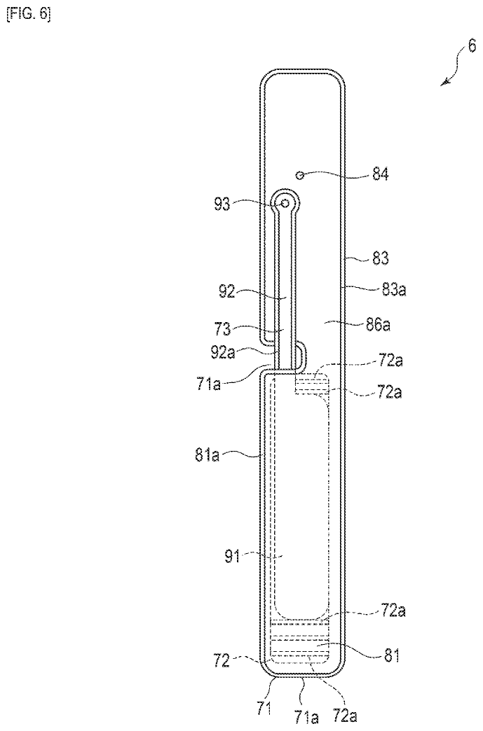

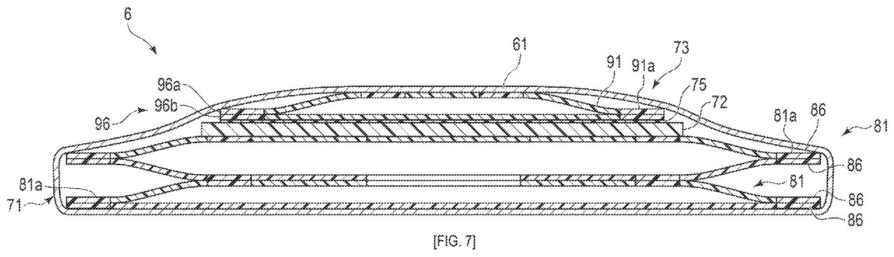

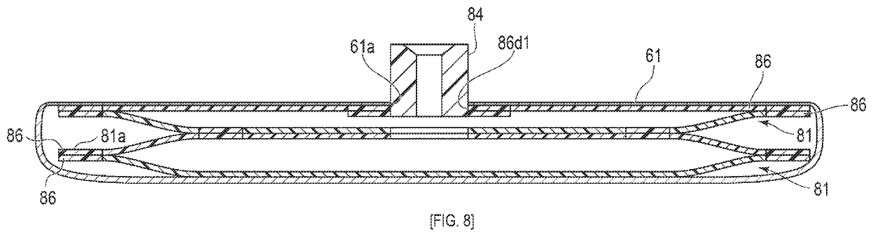

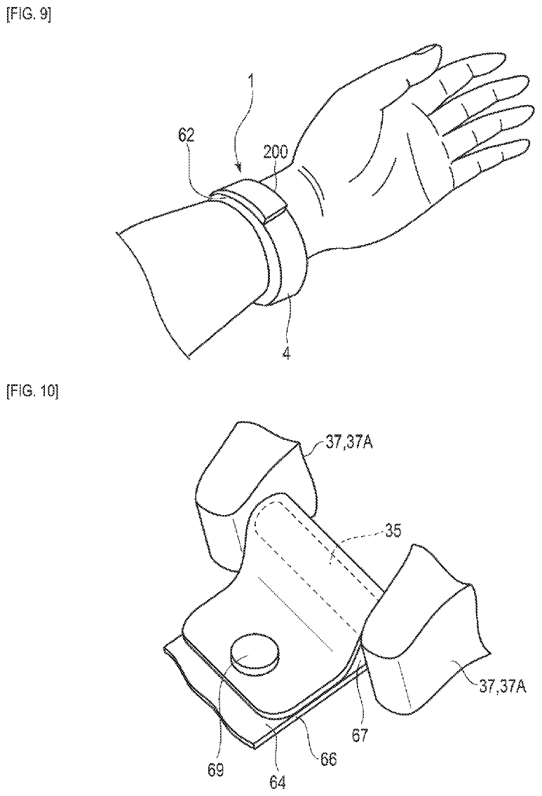

FIG. 6 is a plan view illustrating the configuration of the cuff structure 6. FIG. 7 is a cross-sectional view illustrating the configuration of the belt 4 and the cuff structure 6. Specifically, FIG. 7 illustrates a state in which the belt 4 and the cuff structure 6 are cut along a width direction of the cuff structure 6 at a position passing through a pressing cuff 71, a back plate 72, and a sensing cuff 73. FIG. 8 is a cross-sectional view illustrating the configuration of the belt 4 and the cuff structure 6. Specifically, FIG. 8 illustrates a state in which the belt 4 and the cuff structure 6 are cut along the width direction at a position passing through the pressing cuff 71 and a first connecting portion 84. FIG. 9 is an explanatory diagram illustrating a state in which the blood pressure measurement device 1 is worn on a wrist.

The blood pressure measurement device 1 is an electronic blood pressure measurement device attached to a living body. The present embodiment will be described using an electronic blood pressure measurement device having an aspect of a wearable device attached to the wrist 200 of the living body.

As illustrated in FIG. 1 and FIG. 2, the blood pressure measurement device 1 includes a device body 3, the belt 4 provided at the device body 3 and configured to wind the device body 3 around the wrist 200, and the cuff structure 6 accommodated in the belt 4.

The device body 3 includes, for example, a case 11, a display unit 12, and an operation unit 13. In addition, the device body 3 includes, in the case 11, a pump 14 that inflates the cuff structure 6, a flow path portion that fluidly connects the pump 14 and the cuff structure 6, and a control board.

The case 11 includes an outer case 31 and a windshield 32 that covers an opening of the outer case 31 on a side opposite to the wrist 200 side.

The outer case 31 includes, for example, a case body 33 formed in a tubular shape, two pairs of lugs 37, a bottom wall portion 34 provided at one end of the case body 33, an attached portion 35, and a shaft portion 36.

The case body 33 is formed in, for example, a cylindrical shape.

One pair of lugs 37 is provided at one of symmetrical positions in the circumferential direction of the outer peripheral surface of the case body 33. The other pair of lugs 37 is provided at the other of the symmetrical positions in the circumferential direction of the outer peripheral surface of the case body 33. The pair of lugs 37 are aligned in the circumferential direction of the case body 33. The one pair of lugs 37 are referred to as first lugs 37A, and the other pair of lugs 37 are referred to as second lugs 37B.

The attached portion 35 is provided, for example, between the pair of first lugs 37A. An attachment portion 64, which will be described later, of the belt 4 is attached to the attached portion 35. For example, the attached portion 35 is detachably attached to the pair of first lugs 37A. The attached portion 35 is, for example, a spring rod.

The shaft portion 36 functions as a fulcrum at which a bag body 63, which will be described later, of the belt 4 is folded back. For example, the shaft portion 36 is configured to be capable of folding back the bag body 63 around one axis in the width direction of the bag body 63. For example, the shaft portion 36 is configured to be parallel to a direction orthogonal to a winding direction of the bag body 63 around the wrist 200. For example, the shaft portion 36 is configured in a rod shape provided between the pair of second lugs 37B. The shaft portion 36 is, for example, a spring rod detachably attached to the pair of second lugs 37B.

The bottom wall portion 34 is provided at an opening of the case body 33 on the wrist 200 side and covers the opening. Here, the wrist 200 side is the wrist 200 side in a state where the blood pressure measurement device 1 is worn on the wrist 200.

The bottom wall portion 34 is an example of the wrist 200 side of the case 11. A connected portion 38 is formed on the bottom wall portion 34. The connected portion 38 is fluidly continuous with the pump 14. For example, a flow path continuous with the pump 14 is formed in the case 11, and the connected portion 38 constitutes a part of the flow path. The connected portion 38 is configured to be connectable with connecting portions 84 and 93 of the cuff structure 6, which will be described later.

The number of connected portions 38 is the same as the number of connecting portions 84 and 93 of the cuff structure 6. In the present embodiment, as an example, since the cuff structure 6 includes the two connecting portions 84 and 93, two connected portions 38 are formed. One of the connected portions 38 is referred to as a first connected portion 38A, and the other connected portion 38 is referred to as a second connected portion 38B.

The first connected portion 38A is formed, for example, in a shape into which the connecting portion 84 is inserted. The first connected portion 38A is formed, for example, in a hole shape that is open to an outer surface 34a of the bottom wall portion 34.

The second connected portion 38B is formed, for example, in a shape into which the connecting portion 94 is inserted. The second connected portion 38B is formed, for example, in a hole shape that is open to the outer surface 34a of the bottom wall portion 34.

The windshield 32 is, for example, a circular glass plate.

The display unit 12 is disposed at a position facing the windshield 32. As illustrated in FIG. 1, the display unit 12 is electrically connected to the control board. The display unit 12 is, for example, a liquid crystal display or an organic electroluminescence display. The display unit 12 displays various types of information including the date and time, and measurement results of blood pressure values such as the systolic blood pressure and diastolic blood pressure, heart rate, and the like.

The operation unit 13 is configured to be able to input a command from a user. For example, as illustrated in FIG. 1, the operation unit 13 includes a plurality of buttons 41 provided on the case 11 and a sensor that detects operations of the buttons 41. As the plurality of buttons 41, for example, three buttons are provided.

The belt 4 includes a belt body 61 and a fixing portion 62. The belt body 61 includes the bag body 63 and the attachment portion 64.

The bag body 63 has a bag shape that accommodates the cuff structure 6 therein, and is formed in a band shape that is wound around the wrist 200. Further, the bag body 63 is configured to be able to press the cuff structure 6 against the wrist 200 in a state where the bag body 63 is wound around the wrist 200 and fixed by the fixing portion 62. Further, the bag body 63 is configured to be able to press the inflated cuff structure 6 against the wrist 200 at the time of blood pressure measurement.

In addition, in the present embodiment, the cuff structure 6 is configured to be inflated by air supplied from the pump 14, and the bag body 63 is configured in a bag shape that does not hinder the inflation of the cuff structure 6.

The bag body 63 is set to have a length folded back at the shaft portion 36 in a state where the blood pressure measurement device 1 is worn on the wrist 200, and an inner dimension of the bag body 63 in the longitudinal direction is set to a length of the cuff structure 6 in the circumferential direction (longitudinal direction).

In the present embodiment, the cuff structure 6 includes, for example, the pressing cuff 71, the back plate 72, and the sensing cuff 73. Additionally, for example, a dimension of the pressing cuff 71 in the longitudinal direction is longer than a dimension of each of the back plate 72 and the sensing cuff 73 in the longitudinal direction. Thus, the inner dimension of the bag body 63 in the longitudinal direction is set to be equal to or larger than the length of the pressing cuff 71 in the longitudinal direction.

An inner dimension of the bag body 63 in a lateral direction is set to be equal to or larger than a length of the cuff structure 6 in the lateral direction, and is, for example, substantially equal to a width of the cuff structure 6 in the lateral direction. In the present embodiment, for example, a dimension of the pressing cuff 71 in the lateral direction is longer than a length of each of the back plate 72 and the sensing cuff 73 in the lateral direction. Thus, the inner dimension of the bag body 63 in the lateral direction is substantially equal to the length of the pressing cuff 71 in the lateral direction.

An inner dimension of the bag body 63 in a thickness direction is set to a length that does not inhibit the inflation of the cuff structure 6. For example, the inner dimension of the bag body 63 in the thickness direction is set to a length including an inflation margin of the cuff structure 6.

As illustrated in FIG. 4, holes 65 are formed on one end side in the longitudinal direction of the bag body 63. The number of holes 65 is the same as the number of connecting portions 84 and 93 provided in the cuff structure 6. In the present embodiment, since two connecting portions 84 and 93 are provided, two holes 65 are formed. One hole 65 is referred to as a first hole 65A, and the other hole is referred to as a second hole 65B.

The first hole 65A is formed in a portion facing the first connecting portion 84 in a state where the cuff structure 6 is accommodated in the bag body 63. The first connecting portion 84 is disposed in the first hole 65A in a state where the cuff structure 6 is accommodated in the bag body 63. The second hole 65B is formed at a portion facing a second connecting portion 93 in a state where the cuff structure 6 is accommodated in the bag body 63. The second connecting portion 93 is disposed in the second hole 65B in a state where the cuff structure 6 is accommodated in the bag body 63.

The bag body 63 configured as described above is made of, for example, cloth.

The attachment portion 64 is formed in a band shape, and one end portion thereof is fixed on the bag body 63 side and on the other end side of the bag body 63 from the first hole 65A and the second hole 65B. The other end portion of the attachment portion 64 is attached to the attached portion 35.

A length from the one end portion of the attachment portion 64 to the attached portion 35 is smaller than both a length from the one end portion of the attachment portion 64 to the first connecting portion 84 and a length from the one end portion of the attachment portion 64 to the second connecting portion 93 in a state where the cuff structure 6 is accommodated in the bag body 63.

The attachment portion 64 includes, for example, an attachment portion body 66 formed in a band shape, and a support portion 67 provided at the attachment portion body 66 and supported by the attached portion 35 by disposing the attached portion 35 therein.

One end portion of the attachment portion body 66 in the longitudinal direction is fixed to the bag body 63 in a posture in which a width direction of the attachment portion body 66 is parallel to the width direction of the bag body 63. One end portion of the attachment portion body 66 is fixed to the bag body 63, for example, in a range from one end to the other end in the width direction.

In addition, the attachment portion body 66 is configured to be rotatable around one axis parallel to the width direction of the attachment portion body 66. The attachment portion body 66 has, for example, flexibility, and is bent around one axis parallel to the width direction of the attachment portion body 66 to rotate around the one axis.

The attachment portion body 66 configured as described above is made of, for example, cloth. The one end portion of the attachment portion body 66 is fixed to the bag body 63 by sewing, for example.

The support portion 67 is formed in a tubular shape with both ends being open in the width direction of the attachment portion body 66. The support portion 67 is configured such that a part of the attached portion 35 can be disposed therein. A dimension of the support portion 67 in the axial direction is, for example, the same or substantially the same as a length of the attached portion 35.

The support portion 67 is configured to be rotatable with respect to the attached portion 35. The support portion 67 has, for example, a size that allows the support portion 67 to rotate with respect to the attached portion 35 disposed therein. The support portion 67 is made of, for example, cloth. The support portion 67 is fixed to the attachment portion body 66 by sewing, for example. The support portion 67 may be configured by, for example, folding back and fixing an end portion of the attachment portion body 66.

The fixing portion 62 is configured to be able to fix a portion of the bag body 63 that is folded back at the shaft portion 36 and layered. As illustrated in FIG. 4, the fixing portion 62 is, for example, a surface fastener.

The fixing portion 62 includes, for example, a hook portion 62a provided with a plurality of hooks, and a loop portion 62b provided with a plurality of loops. The hook portion 62a is provided at the other end portion of the bag body 63. The loop portion 62b is provided in a region of the bag body 63 facing the hook portion 62a in a state where the bag body 63 is tightened to the wrist 200.

Note that in the above-described example, in the fixing portion 62, the hook portion 62a and the loop portion 62b are individually configured in different regions on the surface of the bag body 63, but the fixing portion 62 is not limited thereto. In another example, the fixing portion 62 may have a configuration in which a plurality of hooks and a plurality of loops are mixedly provided in the same predetermined region.

The belt 4 configured as described above forms an annular shape together with the device body 3 as illustrated in FIG. 2 by fixing, by using the fixing portion 62, a portion where the other end side of the bag body 63 is folded back at the shaft portion 36 and layered.

The cuff structure 6 is accommodated in the belt 4 as illustrated in FIG. 2. As illustrated in FIG. 2, FIG. 3, and FIG. 5 to FIG. 8, the cuff structure 6 includes, for example, the pressing cuff 71, the back plate 72, and the sensing cuff 73.

In the cuff structure 6, the back plate 72 is disposed on the pressing cuff 71, and the sensing cuff 73 is disposed on the back plate 72. In the cuff structure 6, components disposed adjacent to each other are fixed to each other by using, for example, a joining layer 75. The joining layer 75 is made of, for example, an adhesive or a double-sided tape.

The pressing cuff 71 is configured in a band shape extending in one direction. As illustrated in FIG. 6 and FIG. 7, the pressing cuff 71 includes, for example, a plurality of air bags 81 and the first connecting portion 84 connected to the first connected portion 38A of the device body 3. The pressing cuff 71 is fluidly connected to the pump 14 by the first connecting portion 84 being connected to the first connected portion 38A.

The plurality of air bags 81 are, for example, two air bags 81. The pressing cuff 71 with such a configuration is configured by integrally welding a plurality of sheet members 86 together.

Here, the air bag 81 is a bag-like structure, and in the present embodiment, the blood pressure measurement device 1 is configured to use air with the pump, and thus the present embodiment will be described using the air bag. However, in a case where a fluid other than air is used, the bag-like structure may be a fluid bag that is inflated by the fluid. The plurality of air bags 81 are layered and are in fluid communication with one another in a layering direction.

Each of the air bags 81 is formed in a rectangular bag shape that is long in one direction. The air bag 81 is configured by, for example, combining two sheet members 86 and thermally welding the sheet members 86 in a rectangular frame shape that is long in one direction, as illustrated in a welded portion 81a in FIG. 6.

The first connecting portion 84 is configured to have a shape protruding from the air bag 81 toward outside, for example. Specifically, the first connecting portion 84 is provided in the air bag 81 that faces the bottom wall portion 34 of the case 11 through the belt 4. A tip end of the first connecting portion 84 is exposed from the sheet member 86 on the device body 3 side, of the two sheet members 86 constituting the air bag 81.

The first connecting portion 84 is disposed in the first hole 65A of the bag body 63. The first connecting portion 84 is connected to the first connected portion 38A by being inserted into the first connected portion 38A, for example. The first connecting portion 84 is connected to the first connected portion 38A, and thus the pressing cuff 71 is fluidly connected to the pump 14.

The first connecting portion 84 and the first connected portion 38A may be fixed to each other such that the connection between the first connecting portion 84 and the first connected portion 38A is maintained during blood pressure measurement and the first connecting portion 84 has a holding force enough to maintain the connection with the first connected portion 38A even when the blood pressure measurement device 1 is detached from the wrist 200.

As illustrated in FIG. 5 and FIG. 6, the back plate 72 is formed in a plate shape elongated in one direction. The back plate 72 is stuck to the outer surface of the first sheet member 86a of the pressing cuff 71 by using the joining layer 75. The back plate 72 transfers a pressing force from the pressing cuff 71 to a main surface of the sensing cuff 73 on the back plate 72 side in a state where the back plate 72 is extending along the shape of the wrist 200.

The back plate 72 has shape followability. Here, “shape followability” refers to a function in which the back plate 72 can be deformed in such a manner as to follow the shape of a contacted portion of the wrist 200 to be disposed. This contacted portion of the wrist 200 refers to a region of the wrist 200 that faces the back plate 72. This contact includes both direct contact and indirect contact with the sensing cuff 73 interposed therebetween.

For example, as illustrated in FIG. 6, the back plate 72 includes a plurality of grooves 72a extending in a direction orthogonal to a longitudinal direction of the back plate 72 on both main surfaces of the back plate 72. The plurality of grooves 72a provided on both the main surfaces face each other in the thickness direction of the back plate 72, for example. In the back plate 72, portions including the plurality of grooves 72a are thinner than portions including no grooves 72a and thus the portions including the plurality of grooves 72a are easily deformed. Accordingly, the back plate 72 is deformed in such a manner as to follow the shape of the wrist 200, and has shape followability of extending in the circumferential direction of the wrist 200.

The sensing cuff 73 is fixed to the main surface of the back plate 72 on the wrist 200 side by the joining layer 75. The length of the sensing cuff 73 in the longitudinal direction is set to a length that allows the sensing cuff 73 to be in contact with a region where at least one of a radial artery 211 and an ulnar artery 212, which are the arteries 210 of the wrist 200, exists.

For example, the sensing cuff 73 is formed in the same shape as that of the back plate 72 or a shape that is smaller than that of the back plate 72, in the longitudinal direction and the width direction of the back plate 72. The sensing cuff 73 is inflated to compress a hand palm side region of the wrist 200 in which the artery 210 resides. The sensing cuff 73 is pressed by the inflated pressing cuff 71 toward the wrist 200 side with the back plate 72 interposed therebetween.

The sensing cuff 73 includes the second connecting portion 93 and is fluidly connected to the pump 14 by the second connected portion 93 being connected to the second connected portion 38B.

As illustrated in FIG. 5 to FIG. 7, the sensing cuff 73 includes, for example, one air bag 91, a flow path body 92 that communicates with the air bag 91, and the second connecting portion 93 provided at a tip end of the flow path body 92. One main surface of the air bag 91 of the sensing cuff 73 is fixed to the back plate 72 by using the joining layer 75. For example, the sensing cuff 73 is joined to the main surface of the back plate 72 on the wrist 200 side by the joining layer 75. The sensing cuff 73 with such a configuration is constituted by welding two sheet members 96.

Here, the air bag 91 is a bag-like structure, and in the present embodiment, the blood pressure measurement device 1 is configured to use air with the pump, and thus the present embodiment will be described using the air bag. However, in a case where a fluid other than air is used, the bag-like structure may be any fluid bag that is inflated by the fluid.

The air bag 91 is constituted in a rectangular shape that is long in one direction. The air bag 91 is configured by, for example, combining the two sheet members 96 that are long in one direction and thermally welding the sheet members 96 in a rectangular frame shape that is long in one direction, as illustrated in a welded portion 91a in FIG. 7.

The flow path body 92 is integrally provided at a part of one edge of the air bag 91 in the longitudinal direction. As a specific example, the flow path body 92 is provided at the end portion of the air bag 91 near the device body 3. Additionally, the flow path body 92 is formed in a shape that is long in one direction and has less width than a width of the air bag 91 in a lateral direction, and formed with a tip end having a circular shape. The flow path body 92 includes the second connecting portion 93 on the tip end thereof. The flow path body 92 is connected to a flow path portion through the second connecting portion 93 and constitutes a flow path between the flow path portion of the device body 3 and the air bag 91.

The flow path body 92 is configured by thermally welding a part of the sheet members 96 that are adjacent to a region of the sheet members 96 constituting the air bag 91, in a frame shape that is long in one direction, in a state where the second connecting portion 93 is disposed between the two sheet members 96. Note that, a part of the welded portion 91a where the two sheet members 96 are welded in the rectangular frame shape is not welded and the air bag 91 is configured to be continuous with a welded portion 92a constituting the flow path body 92, and thus the air bag 91 and the flow path body 92 fluidly communicate with each other.

The second connecting portion 93 is configured in a shape protruding from the air bag 91 toward outside, for example. The second connecting portion 93 is provided at the tip end of the flow path body 92. Also, the tip end of the second connecting portion 93 is externally exposed from the sheet member 96 facing the back plate 72, of the two sheet members 96 constituting the flow path body 92.

The second connecting portion 93 is disposed in the second hole 65B of the bag body 63. The second connecting portion 93 is connected to the second connected portion 38B by being inserted into the second connected portion 38B, for example. The second connecting portion 93 is connected to the second connected portion 38B, and thus the sensing cuff 73 is fluidly connected to the pump 14.

The second connecting portion 93 and the second connected portion 38B may be fixed to each other such that the connection between the second connecting portion 93 and the second connected portion 38B is maintained during blood pressure measurement and the second connecting portion 93 has a holding force enough to maintain the connection with the second connected portion 38B even when the blood pressure measurement device 1 is detached from the wrist 200.

The cuff structure 6 configured as described above is provided with a configuration in which the second connecting portion 93 of the sensing cuff 73 is disposed on the first connecting portion 84 side of the pressing cuff 71 in the thickness direction of the cuff structure 6. As an example of the configuration, as illustrated in FIG. 6, the pressing cuff 71 includes an insertion portion 71a that allows a part of the sensing cuff 73 at, for example, a middle portion of the pressing cuff 71 in the longitudinal direction to be disposed. The insertion portion 71a is formed, for example, in a shape in which a part of an edge portion of the pressing cuff 71 along the longitudinal direction is recessed in the lateral direction of the pressing cuff 71. The insertion portion 71a is configured to allow the flow path body 92 of the sensing cuff 73 to pass from the inner circumferential surface side of the pressing cuff 71 to the device body 3 side. Note that the shape of the insertion portion 71a is not limited to the shape in which the part of the edge portion of the pressing cuff 71 along the longitudinal direction is recessed in the lateral direction of the pressing cuff 71. In another example, the insertion portion 71a may be a hole in which the second connecting portion 93 can be disposed. Alternatively, the flow path body 92 may be configured in a shape extending to the first connecting portion 84 side beyond the edge of the pressing cuff 71 along the longitudinal direction.

Next, as illustrated in FIG. 9, an example in which the user wears the blood pressure measurement device 1 on the wrist 200 will be described.

First, the user peels off the hook portion 101 fixed to the loop portion 102 from the loop portion 102 and pulls out the bag body 63 from between the shaft portion 36 and the outer case 31 to adjust an annular space formed by the bag body 63 and the device body 3 to a size into which the wrist 200 can be inserted.

Next, the user inserts the wrist 200 into the annular space formed by the bag body 63 and the device body 3.

Next, the user moves the blood pressure measurement device 1 with respect to the wrist 200, and thus the sensing cuff 73 faces a region of the wrist 200 where the artery is present.

Next, the user presses the sensing cuff 73 against the region of the wrist 200 where the artery exists by pulling the end portion of the bag body 63 on the hook portion 101 side and thus tightening the bag body 63, and fixes the hook portion 101 to the loop portion 102. The blood pressure measurement device 1 is thus worn on the wrist 200 by this procedure.

In the blood pressure measurement device 1 configured as described above, the cuff structure 6 is accommodated in the bag body 63 of the belt body 61 of the belt 4. The bag body 63 does not inhibit the inflation of the cuff structure 6. In addition, the bag body 63 presses the cuff structure 6 against the wrist 200 in a state of being wounded around the wrist 200 and fixed by the fixing portion 62.

Thus, when the blood pressure measurement device 1 is worn on the wrist 200, even when the cuff structure 6 moves in the axial direction of the wrist 200 with respect to the bag body 63, the cuff structure 6 is accommodated in the bag body 63, so that the cuff structure 6 can be suitably pressed against the wrist 200. As a result, measurement accuracy of a blood pressure can be improved.

Further, the cuff structure 6 is fluidly connected to the pump 14 by the connections of the connecting portions 84 and 93 and the connected portions 38A and 38B, and is mechanically attached to the device body 3 by the attachment portion 64 of the belt 4 being attached to the attached portion 35 of the device body 3. For this reason, fixing of the connecting portions 84 and 93 and the connected portions 38A and 38B only needs to have a holding force enough to maintain the fluid connection between the cuff structure 6 and the pump 14, and a configuration for directly attaching the cuff structure 6 to the device body 3 is not necessary. Thus, the configuration of the blood pressure measurement device 1 can be simplified. For this reason, for example, the connecting portions 84 and 93 and the connected portions 38A and 38B can have a simple configuration in which the connecting portions 84 and 93 are connected to the connected portions 38A and 38B only by inserting the connecting portions 84 and 93 into the connected portions 38A and 38B.

Further, a length from the one end portion of the attachment portion 64 fixed to the bag body 63 to the attached portion 35 is shorter than lengths from the one end portion of the attachment portion 64 fixed to the bag body 63 to both of the connecting portions 84 and 93. For this reason, even when the user pulls the bag body 63 when the blood pressure measurement device 1 is worn on the wrist 200 or the like, a tensile force does not act between the one end of the bag body 63 and the one end portion of the attachment portion 64. Thus, since the connecting portions 84 and 93 are not pressed by edges of the holes 65A and 65B of the bag body 63, the connecting portions 84 and 93 do not press the connected portions 38A and 38B in a direction intersecting the direction in which the connecting portions 84 and 93 protrude.

Further, the connecting portions 84 and 93 and the connected portions 38A and 38B have a configuration in which one is inserted into the other. Thus, the connecting portions 84 and 93 can be easily connected to the connected portions 38A and 38B.

Further, since the connected portions 38A and 38B are provided on the wrist 200 side of the case 11, in a state where the blood pressure measurement device 1 is worn on the wrist 200, movement of the connecting portions 84 and 93 in a direction in which the connecting portions 84 and 93 come out from the connected portions 38A and 38B is restricted by the wrist 200.

Thus, the holding force for holding the state in which the connecting portions 84 and 93 are inserted into the connected portions 38A and 38B may be such a holding force enough to prevent the connecting portions 84 and 93 from becoming disconnected from the connected portions 38A and 38B in the state in which the blood pressure measurement device 1 is removed from the wrist 200.

Further, since the attachment portion body 66 has flexibility, a movable region of the belt 4 around the spring rod 35 can be increased. Thus, the blood pressure measurement device 1 can be easily attached and detached to and from the wrist 200.

Furthermore, using a surface fastener as the fixing portion 62 that fixes the bag body 63 wound around the wrist 200 allows a length of the bag body 63 along the circumferential direction of the wrist 200 to be adjusted in a stepless manner in using the blood pressure measurement device 1.

As described above, according to the blood pressure measurement device 1 according to the present embodiment, the blood pressure measurement device 1 in which the measurement accuracy of the blood pressure can be prevented from deteriorating can be provided.

Note that in the above-described example, the configuration in which the blood pressure measurement device 1 includes the belt 4 made of cloth has been described as an example, but the present invention is not limited thereto. In another example, the belt 4 may be formed using resin as a material.

Furthermore, in the above-described example, the configuration where the attachment portion 64 is removed from the device body 3 by removing the attached portion 35 from the device body 3 has been described as an example, but the present invention is not limited thereto. In another example, the attachment portion 64 may be configured to be detachably attached to the attached portion 35. For example, as in a modified example illustrated in FIG. 10, the support portion 67 may be configured by folding back the end portion of the attachment portion body 66 on the device body 3 side and fixing the folded back portion to another portion of the attachment portion body 66 by using a detachable fixing portion 69 such as a button. For example, a surface fastener may be used as the fixing portion 69 instead of the button.

Since the support portion 67 is configured as described above, when the belt 4 is removed from the device body 3, the bag body 63 can be removed from the device body 3 without removing the attached portion 35 from the pair of first lugs 37A by releasing the fixing by the detachable fixing portion 69. Thus, it is possible to improve the efficiency of the operation of attaching and detaching the belt 4 to and from the device body 3.

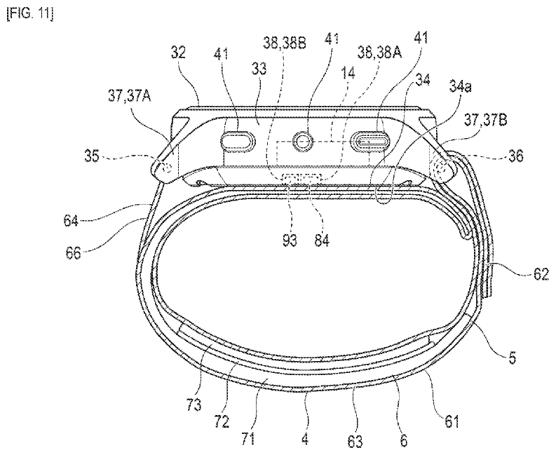

In addition, as a modified example illustrated in FIG. 11, the blood pressure measurement device 1 may further include a curler 5. The curler 5 is configured in a band shape whose one end and the other end are separated from each other and that curves following along the circumferential direction of the wrist 200. In the curler 5, the cuff structure 6 is disposed on the inner circumferential surface. The curler 5 holds a part of the cuff structure 6 along the shape of the inner circumferential surface of the curler 5. The curler 5 is accommodated in the belt 4.

For example, the curler 5 holds the cuff structure 6 by fixing the cuff structure 6 by a joining layer provided between the curler 5 and the cuff structure 6. The curler 5 is formed with a hole in which the first connecting portion 84 is disposed and a hole in which the second connecting portion 93 is disposed.

Further, the curler 5 has a hardness appropriate to provide flexibility and shape retainability. Here, the “flexibility” refers to deformation of the shape of the curler 5 in a radial direction at the time of application of an external force due to the belt 4 to the curler 5. For example, the “flexibility” refers to deformation of the shape of the curler 5 in a side view in which the curler 5 approaches the wrist 200, is along the shape of the wrist 200, or follows the shape of the wrist 200 when the curler 5 is pressed by the belt 4. Furthermore, the “shape retainability” refers to the ability of the curler to maintain a shape imparted in advance when no external force is applied to the curler. For example, the “shape retainability” refers to, in the present embodiment, the ability of the curler 5 to maintain the shape curving along the circumferential direction of the wrist 200.

Further, in the above-described example, the configuration in which each of the connecting portions 84 and 93 is formed in a protruding shape protruding toward outside has been described as an example, but the present invention is not limited thereto. For example, a configuration may be adopted in which the connected portions 38A and 38B formed in the device body 3 are formed in a shape protruding from the bottom wall portion 34, the connecting portions 84 and 93 are formed in a hole shape, and the connected portions 38A and 38B are inserted into the connecting portions 84 and 93 to connect the connecting portions 84 and 93 and the connected portions 38A and 38B to each other.

Furthermore, in the above-described example, the configuration where the belt 4 is folded back at the shaft portion 36 and fixed at the fixing portion 62 has been described as an example, but the present invention is not limited thereto. In another example, the bag body 63 and the attachment portion 64 may be used as one belt member of a belt having a configuration in which belt members fixed at respective symmetrical positions in the circumferential direction of the outer circumferential surface of the device body 3 are provided and the belt members are connected to each other by using, for example, a buckle.

Further, in the example described above, as the configuration in which, of the connecting portions 84 and 93 and the connected portions 38A and 38B, one is inserted into the other, the configuration in which each of the connecting portions 84 and 93 is formed in a protruding shape and is inserted into the corresponding connected portions 38A and 38B has been described as an example, but the configuration is not limited thereto. In another example, the connected portions 38A and 38B may be inserted into the connecting portions 84 and 93.

That is, the present invention is not limited to the above-described embodiment, and various modifications can be made in an implementation stage without departing from the gist thereof. Furthermore, each of the embodiments may be carried out as appropriate in a combination as much as possible, and combined effects can be obtained in such a case. Furthermore, the inventions at various stages are included in the above-described embodiment, and the various inventions can be extracted in accordance with appropriate combinations in the plurality of disclosed constituent elements. For example, in a case where the problem can be solved and the effects can be obtained even when some constituent elements are removed from the entire constituent elements given in the embodiment, the configuration obtained by removing the constituent elements may be extracted as an invention.

### REFERENCE NUMERALS LIST

1 Blood pressure measurement device
3 Device body
4 Belt
5 Curler
6 Cuff structure
7 Attachment portion
8 Fixing portion
11 Case
12 Display unit
13 Operation unit
14 Pump
31 Outer case
32 Windshield
33 Case body
34 Bottom wall portion
34a Outer surface
35 Attached portion
36 Shaft portion
37 Lug
37A First lug
37B Second lug
38 Connected portion
38A First connected portion
38B Second connected portion
41 Button
61 Belt body
62 Fixing portion
62a Hook portion
62b Loop portion
63 Bag body
64 Attachment portion
65 Hole
65A First hole
65B Second hole
66 Attachment portion body
67 Support portion
69 Fixing portion
71 Pressing cuff
71a Insertion portion
72 Back plate
72a Groove
73 Sensing cuff
75 Joining layer
81 Air bag
81a Welded portion
81b Bridge welded portion
84 Connecting portion
86 Sheet member
91 Air bag
91a Welded portion
92 Flow path body
92a Welded portion
93 Connecting portion
96 Sheet member
101 Hook portion
102 Loop portion
200 Wrist
210 Artery
211 Radial artery
212 Ulnar artery

1. A blood pressure measurement device to be worn on a wrist of a user, the blood pressure measurement device comprising:
a case including a case body, an attached portion provided at one of symmetrical positions in a circumferential direction of an outer peripheral surface of the case body, a shaft portion provided at the other symmetrical position, and a connected portion provided on a wrist side of the case body;
a pump accommodated in the case body, the pump being fluidly continuous with the connected portion;
a cuff including a connecting portion provided at a portion facing the wrist side of the case body, the connecting portion being connected to the connected portion, the cuff being fluidly connected to the pump through the connecting portion; and
a belt including a bag body inside which the cuff is accommodated, the bag body having a band shape to be wound around the wrist, the bag body being formed with a hole disposed with the connecting portion on one end side in a longitudinal direction, an attachment portion including one end portion fixed on the one end side of the bag body and the other end side from the hole, and the other end portion attached to the attached portion, the attachment portion having a length from the one end portion to the attached portion shorter than a length from the one end portion to the connecting portion disposed in the hole, and a fixing portion provided at the bag body, the fixing portion being configured to fix a portion configured to be folded back and layered at the shaft portion of the bag body.

2. The blood pressure measurement device according to claim 1, wherein
one of the connected portion and the connecting portion is inserted into the other of the connected portion and the connecting portion.

3. The blood pressure measurement device according to claim 1, wherein
the attachment portion has flexibility.

4. The blood pressure measurement device according to claim 2, wherein
the bag body and the attachment portion are made of cloth.
